(19) United States
(12) Patent Application Publication
(10) Pub. No.: US 2022/0308147 A1
(43) Pub. Date: Sep. 29, 2022

(54) ENHANCEMENTS TO QUANTITATIVE MAGNETIC RESONANCE IMAGING TECHNIQUES

(71) Applicant: The University of North Carolina at Chapel Hill, Chapel Hill, NC (US)

(72) Inventors: Yong CHEN, Chapel Hill, NC (US); Zhenghan FANG, Chapel Hill, NC (US); Weili LIN, Chapel Hill, NC (US); Dinggang SHEN, Chapel Hill, NC (US); Pew-Thian YAP, Chapel Hill, NC (US)

(21) Appl. No.: 17/619,046

(22) PCT Filed: Jun. 12, 2020
(86) PCT No.: PCT/US2020/037490

§ 371 Date: Dec. 14, 2021

Related U.S. Application Data
(60) Provisional application No. 62/861,463, filed on Jun. 14, 2019.

(51) Int. Cl. G01R 33/56; G01R 33/50

(52) U.S. Cl. CPC G01R 33/5608; G01R 33/50; G01R 33/5601

(57) ABSTRACT

Systems and methods providing enhancements to quantitative imaging systems and techniques are described herein. In one aspect, a system for tissue quantification in magnetic resonance fingerprinting (MRF) comprises a feature extraction module operable to convert pixel input high-dimensional signal evolution in to a low-dimensional feature map. The system also comprises a spatially constrained quantification module operable to capture spatial information from the low-dimensional feature map and generate an estimated tissue property map.

### RELATED APPLICATION DATA

The present application claims priority pursuant to Article 8 of the Patent Cooperation Treaty to U.S. Provisional Patent Application Ser. No. 62/861,463 filed Jun. 14, 2019 which is incorporated herein by reference in its entirety.

### FIELD

The present application addresses quantitative magnetic resonance techniques and, in particular, to various enhancements to quantitative magnetic resonance techniques for improving patient diagnosis and care.

### BACKGROUND

Quantitative imaging, i.e., quantification of important issue properties in human body such as the T1 and T2 relaxation times, is desired in both clinical and research areas. Compared to the qualitative imaging techniques, e.g., T1- and T2-weighted imaging, quantitative imaging can provide more accurate and unbiased information of the inner body and make it easier to objectively compare different examinations in longitudinal studies. However, one of the major barriers of translating conventional quantitative imaging techniques to clinical applications is the prohibitively long time for data acquisition. Such time delay can render these techniques unsuitable for certain patients and frustrate derivative techniques dependent on the data acquisition.

### SUMMARY

In view of the foregoing, systems and methods providing enhancements to quantitative imaging systems and techniques are described herein. In one aspect, a system for tissue quantification in magnetic resonance fingerprinting (MRF) comprises a feature extraction module operable to convert pixel input high-dimensional signal evolution in to a low-dimensional feature map. The system also comprises a spatially constrained quantification module operable to capture spatial information from the low-dimensional feature map and generate an estimated tissue property map. In some embodiments, the feature extraction module is applied to all pixels in an axial slice or other imaging orientation to generate the low-dimensional feature map corresponding to the high dimensional MRF signals.

In another aspect, methods for tissue quantification in MRF are provided. A method for tissue quantification in MRF, for example, comprises providing pixel input high-dimensional signal evolution to a feature extraction module to generate a low-dimensional feature map, and transferring the low dimension feature map to a spatially constrained quantification module for capturing spatial information form the low-dimensional feature map and generating an estimated tissue property map.

In another aspect, methods of generating synthetic magnetic resonance images employing quantitative magnetic resonance imaging data are described herein. In some embodiments, a method comprises identifying imaging parameters affecting tissue contrast for a type of magnetic resonance image and establishing Bloch equation simulations based on specific pulse sequence structure for the type of magnetic resonance image. Tissue intrinsic parameters are extracted from quantitative tissue maps acquired from a patient via a quantitative magnetic resonance imaging technique, and the synthetic magnetic resonance image is generated using the tissue intrinsic parameters in conjunction with the Bloch equation simulations. In some embodiments, the tissue intrinsic parameters and Bloch equation simulations are employed to simultaneously optimize all imaging parameters to achieve maximal contrast between the differing tissue types in the simulated imaging process employed to construct the synthetic magnetic resonance image. Tissue intrinsic parameters can include, but are not limited to, T1, T2 and spin density (M0). Moreover, the maximal contrast can be between healthy tissue and abnormal tissue.

In a further aspect, methods of three-dimension magnetic resonance fingerprinting are described herein. Briefly, a method of three-dimensional magnetic resonance fingerprinting (MRF) comprises accelerating acquisition of a MRF dataset via application of parallel imaging along the partition-encoding direction, and integrating a convolutional neural network with MRF framework to extract an increased number of parameters from the MRF dataset yielding accelerated tissue mapping and one or more improvements to tissue characterization.

These and other embodiments are further detailed in the following detailed description.

### BRIEF DESCRIPTION OF THE DRAWINGS

FIG. 1 illustrates a two-step deep learning model for spatially constrained tissue quantification in MRF, according to some embodiments.

FIG. 2 illustrates network structure of a fully-connected neural network (FNN) in the feature extraction module according to some embodiments.

FIG. 3 illustrates network structure of a convolutional neural network (CNN) in the spatially constrained quantification module (SQ), according to some embodiments.

FIGS. 4A and 4B illustrate quantification errors in T1 and T2 yielded by the SCQ method and baseline method (DM) for different acceleration rates according to some embodiments.

FIG. 5 provides representative synthetic T₁-weighted, T₂-weighted, bSSFP and DIR images using T₁, T₂, and proton density (Mₒ) maps acquired by MRF according to some embodiments.

FIG. 6 provides synthetic T2w images generated using T₁, T₂ and M₀ maps acquired from a pediatric subject illustrating enhances white/gray contrast according to some embodiments.

FIG. 7 illustrates GRAPPA reconstruction along the partition edge direction according to some embodiments.

FIG. 8A illustrates standard 2DMRF sequence, where pseudorandom acquisition parameters, such as the flip angles (FA), were applied in the 3DMRF acquisition.

FIG. 8B illustrates standard 3DMRF sequence with N time frames. A 2-second waiting time was applied after data acquisition of each partition for partial longitudinal relaxation.

FIG. 8C illustrates 3DMRF sequence for acquiring a training dataset for deep learning, according to some embodiments.

FIG. 9 illustrates an overview of a CNN model with two modules for tissue property mapping according to some embodiments.

FIG. 10 provides comparison of NRMSE values for T₁ and T₂ quantification between three different post-processing methods.

FIG. 11 shows representative T₁ and T₂ maps obtained using the methods described herein with different numbers of time points.

FIG. 12A are representative T₁ and T₂ maps obtained using 3DMRF and 3DMRF-DL sequences relative to the results of reference scans.

FIG. 12B is a comparison of quantitative T₁ and T₂ values between the reference and 3DMRF methods.

FIG. 13 are representative T₁ and T₂ maps using the prospectively accelerated scans with R=2 and 192 points.

FIG. 14 provides reformatted quantitative maps in axial, coronal, and sagittal views from the prospectively accelerated scan (R=2; 192 time points).

FIG. 15 provides representative brain segmentations results from MRF measurements according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### I. Systems and Methods of Tissue Quantification in MRF

In one aspect, a system for tissue quantification in magnetic resonance fingerprinting (MRF) comprises a feature extraction module operable to convert pixel input high-dimensional signal evolution in to a low-dimensional feature map. The system also comprises a spatially constrained quantification module operable to capture spatial information from the low-dimensional feature map and generate an estimated tissue property map. In some embodiments, the feature extraction module is applied to all pixels in an axial slice or other imaging orientation to generate the low-dimensional feature map corresponding to the high dimensional MRF signals.

In another aspect, methods for tissue quantification in MRF are provided. A method for tissue quantification in MRF, for example, comprises providing pixel input high-dimensional signal evolution to a feature extraction module to generate a low-dimensional feature map, and transferring the low dimension feature map to a spatially constrained quantification module for capturing spatial information form the low-dimensional feature map and generating an estimated tissue property map.

Systems and methods described herein exploit spatial context information by using a deep learning model to learn the mapping from the signals at multiple neighboring pixels to the tissue properties at the central pixel. This spatial context information can be helpful for accurate quantification for two reasons. First, the tissue properties at different pixels are not independent, but correlated. For example, the adjacent pixels of one tissue are likely to have similar tissue properties. Therefore, neighboring pixels are used together as spatial constraint to regularize the estimation at the central target pixel and correct possible errors. Second, the undersampling in k-space in MRF acquisition will result in aliasing in the image space, due to distribution of the target pixel signal to neighboring pixels. Therefore, using spatial information may help retrieve the scattered signals and finally provide a better quantification with MRF.

The major difficulty in using deep learning models to exploit spatial information in MRF signals is the high dimension of the observed signal evolution at each pixel due to the large number of time points. To overcome this difficulty, systems and methods described herein introduce a unique two-step deep learning model, with a feature extraction module that reduces the dimension of signals by extracting a low-dimensional feature vector from each high-dimensional signal evolution, followed by a spatially-constrained quantification module that exploits the spatial information from the extracted feature map to generate the final tissue property map. A two-step training strategy is also designed to enhance this two-step model. Moreover, a special relative-difference-based loss function is adopted to tackle with the large quantitative range of the tissue properties to be estimated.

MRF data of cross-section slices of human brain was acquired on a Siemens 3T Prisma scanner using a 32-channel head coil. Highly-undersampled 2DMR images were acquired using the fast imaging with steady state precession (FISP) sequence. For each slice, 2,304 time points were acquired and each time point consists of data from only one spiral readout (reduction factor=48). Other imaging parameters included: field of view (FOV): 30 cm; matrix size: 256×256; slice thickness: 5 mm; flip angle: 5-12°. The MRF dictionary contains 13,123 combinations of T1 (60-5000 ms) and T2 (10-500 ms). The signal evolution corresponding to each combination was simulated by using the Bloch equations. The ground truth tissue property maps were obtained from the acquired MRF data of all 2,304 time points by using the dictionary matching method in the original framework. Specifically, first, MR images are reconstructed by applying the inverse Fourier transform to the zero-filled and Cartesian resampled k-space data. Next, the signal evolution in the dictionary that best matches the observed signal evolution at a certain pixel is selected by using the cross correlation as similarity metric. Then, the T1 and T2 values corresponding to the best-matching entry are assigned to that pixel. The obtained tissue property maps are used as the ground truth in the following experiments.

Since the magnitude of MRF signal evolutions varies largely across different subjects, it is important to normalize the magnitude of signals to a common range for better generalization of the deep learning model. In this work, it was proposed to normalize the energy (i.e., sum of squared magnitude) of the acquired signal evolution at each pixel to 1, which is similar to the normalization performed in calculating cross correlation in the dictionary method.

For simplicity, denote normalizes MRF signals of an axial slice as X∈C^{M×N×T}, where M×N is the size of the imaging matrix, i.e. 256×256 in this study, and T is the number of time points at pixel (m,n) as x_{m·n}∈C^{T}. Denote the ground truth tissue property (T1 or T2) map of the axial slice as θ∈R^{m×N×1}.

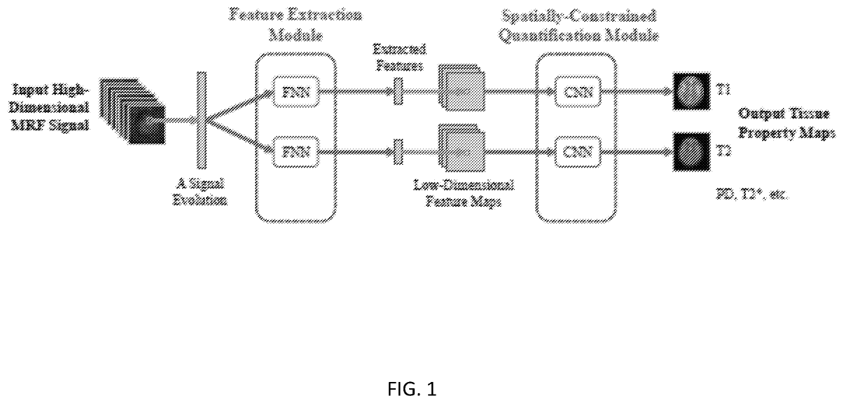

A two-step deep learning model was designed to learn the mapping from the MRF signals X to the tissue property map θ. The model has a feature extraction (FE) module which reduces the dimension of signal evolutions, followed by a spatially-constrained quantification (SQ) module which estimates the tissue property maps from the extracted feature map. The schematic overview of our model is shown in FIG. 1. The structures of the FE and SQ modules are described in details in the following sections.

In the feature extraction (FE) module, a fully-connected neural network (FNN) is used to convert the input high-dimensional signal evolution into a low-dimensional feature vector which contains useful information for tissue property estimation. One network is used for each tissue property to be measured. Specifically, each FNN learns a nonlinear mapping f from the signal evolution at a certain pixel y_{m,n}∈R^{D}:

y_{m,n}=f(x_{m,n})

where D is the number of featured extracted. Applying the FE module to all pixels in the axial slice yields a low-dimensional feature map Y∈R^{M×N×D} corresponding to the high-dimensional MRF signals X:

Y=f(X)

The FE module is needed for the following reasons. First, MRF implementation usually acquires a large number of time points, such as 2,304 in the FISP sequence and 576 after 4 times of acceleration. In this case, it was unreasonable to feed such high-dimensional data directly into the subsequent spatially-constrained quantification module, as it results in a prohibitively large network size that is challenging for training and generalization of the neural networks. Moreover, the FE module can provide a better representation for the original signal by extracting only the useful information for the estimation of the target tissue property while filtering out the noise and unrelated information in the original signal.

Several advantages exist of using deep neural networks in the present analysis. First, the deep neural network learns a multilayer nonlinear mapping from the signal to the extracted features, whereas singular value decomposition (SVD) learns only a singlelayer linear mapping, therefore the deep neural network can extract more abstract and higher-level information from the input signal, which is good for the robustness and accuracy of tissue quantification. Second, using neural networks in both FE and SQ modules allows end-to-end training of the entire two-step model. The end-to-end training can improve the compatibility between two modules and thus the performance of systems and methods described herein.

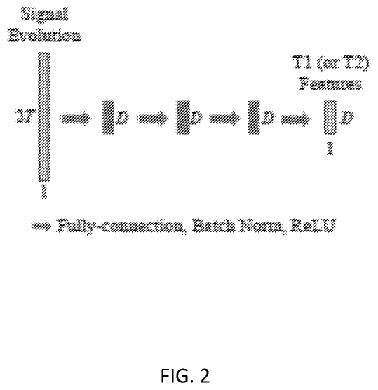

There are various choices for the structure of FNN in the FE module. The structure used in this study is shown in FIG. 2. As shown in FIG. 2, the FNN is composed of 4 fully-connected (FC) layers, where each FC layer has a linear projection followed by batch normalization and ReLU activation. The output dimensions of all the FC layers are the same. The input of FFN, i.e., a signal evolution x_{m,n}∈C^{T}, is transformed into a real vector by splitting the real and imaginary parts, they the input dimension of FNN is 2T.

In the spatially-constrained quantification (SQ) module, a convolutional neural network (CNN) is used to capture spatial information of the feature map Y and finally generate the estimated tissue property map \hat{Θ}∈R^{M×N×1}. One network is used for each tissue property to be measured. Specifically, each CNN learns a nonlinear mapping s from the feature map Y to the estimated tissue property \hat{Θ}:

\hat{Θ}=s(Y)

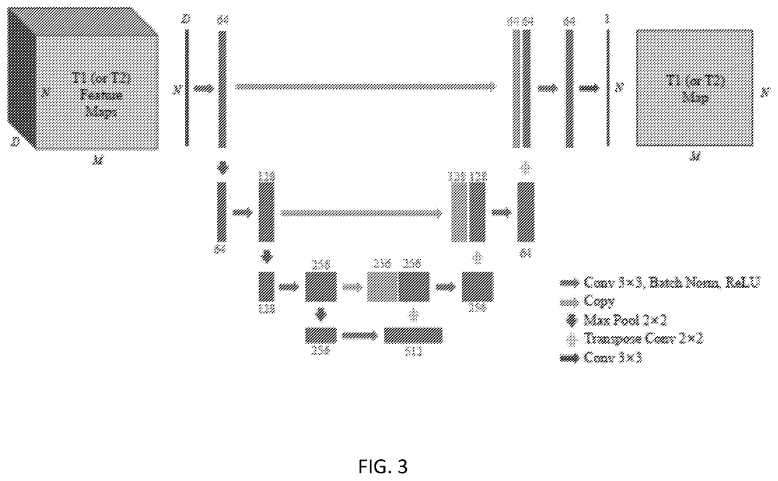

There are various choices for the structure of CNN in the SQ module. In this example, U-Net was employed to capture both the local and global spatial information of feature map Y, with the network structure shown in FIG. 3. As shown in FIG. 3, this network consists of an encoder sub-network (i.e., left part of FIG. 3) that extracts multi-scale spatial features from the input, and a successive decoder sub-network (i.e., right part of FIG. 3) that uses the extracted spatial features to generate the output tissue property (T1 or T2) map. During alternate feature extraction (3×3 convolution followed by ReLU activation) and down-sampling (2×2 max pooling) operations in the encoder sub-network, the information from distributed signals due to aliasing in MRF images is retrieved, and the spatial constraints among different pixels are now implicitly incorporated into the extracted spatial feature maps. Then, the decoder sub-network combines spatial information from different scales by up-sampling (transpose convolution 2×2), copying, and concatenating, to fuse global context knowledge with complementary local details for spatially constrained accurate tissue quantification.

To better train the proposed two-step model, a two-step training strategy was designed which includes 1) pretraining of the FE module by signals and tissue properties at individual pixels and 2) end-to-end training of the entire model by signals and tissue property maps of whole axial slices. Each FNN was extended with one FC layer to output the desired tissue property corresponding to the input signal evolution. Therefore, the features extracted by the original FNN will capture useful information for the quantification of the desired tissue property. Denote the mapping learned by the added FC layer as fₐ and the mapping learned by the extended FNN as fₐ∘f. The pretraining process can be formulated as the following optimization problem:

$${ξ}_{f} , {ξ}_{{f}_{a}} = arg {min}_{{ξ}_{f} , {ξ}_{{f}_{a}}} 𝔼 [ \vert \frac{{θ}_{m , n} - {f}_{a} ∘ f ( {x}_{m , n} )}{{θ}_{m , n}} \vert ]$$

where θ_{m·n}∈R¹ is the ground truth tissue property at pixel m,n, fₐ∘f(x_{m,n)∈R}¹ is the output of the extended FNN for input x_{m,n}, ξ_{f} and ξₐ are network parameters of the original FNN and the added FC layer respectively, and [symbol] [ ] represents the mathematical expectation.

Note that the relative difference was used between the ground truth property at pixel (m,n), fₐ∘f(x_{m,n)∈R}¹ as the loss function, instead of the absolute difference which is commonly used for regression problems. The reason is that T1 and T2 measures in human body have very large quantitative ranges, thus the loss function based on the conventional absolute difference will be dominated by the tissues with high T1 or T2 values. Therefore, the relative difference was used as the loss function to balance over the tissues with different property ranges.

The pretraining of FE module is helpful in two ways. First, it provides better initial parameters of the FE module for the following end-to-end training of the entire model. Second, during pretraining, more data can be used to better train the FE module since the signal and tissue property at each individual pixel can be used as training data. In contrast, during end-to end training, the signals and tissue properties of whole slices or patches must be used as training data to provide spatial context information for the SQ module.

After the pretraining, end-to-end training is performed to train the SQ module and fine-tune the FE module. During the end-to-end training, the parameters in both the FE and SQ modules are tuned together, so that the two modules are more compatible and the performance of the entire model is improved. The end-to-end training can be formulated as the following optimization problem:

$${ξ}_{s} , {ξ}_{f} = arg {min}_{{ξ}_{s} , {ξ}_{f}} 𝔼 [ {‖ \frac{Θ - s ∘ f ( X )}{Θ} ‖}_{1} ]$$

where s and f are the mappings learned by the SQ and FE modules respectively, ξₛ and ξ_{f} are the network parameters of the SQ and FE modules respectively, s∘f(X)∈R^{M×N×1} is the output of the entire model for input X, and ∥·∥₁ stands for the entry-wise 1-norm. The optimization problems in both the pretraining and end-to-end training are solved by the stochastic gradient descent method with ADAM optimizer. The training algorithm is implemented in PyTorch 0.2.0_4 and run on a GeForce GTC TITAN XP GPU.

When the training is completed, the model can be applied on new data for tissue quantification. Specifically, the model can calculate the desired tissue property map \hat{Θ}∈R^{M×N×1} for the input MRF signals of an axial slice X∈R^{M×N×1} by:

\hat{Θ}=s∘f(X)

Note that the tissue quantification is performed by a direct mapping from the observed signals to the tissue property map. Accordingly, systems and methods described herein are more computationally efficient than dictionary-based and model-based methods requiring iterative computations.

Performance of systems and methods described herein was tested according to the following parameters. A dataset was employed containing axial slices from 6 human subjects. For 5 subjects, 12 slices were acquired per subject and for the other 1 subject, 10 slices were acquired. For the learning-based methods, the slices from 5 subjects were used as training data, and those from the remaining 1 subject were used as test data. In the experiments that do not perform cross validation, a fixed subject (n=5) was used as the test data.

MRF acquisition data was accelerated by using fewer time points for tissue quantification. For the acceleration rate ar, only the first

$$\frac{1}{ar} \cdot {T}_{a}$$

of all T^{a} (i.e. 2,304) time points were used. For example, when ar=4, only the first ¼×2304=576 times points were used to estimate the tissue properties, i.e., T=576.

Relative error was used to measure the quantification accuracy:

e_{m,n}=|(θ_{m,n}−\hat{θ}_{m,n})/θ_{m,n}| where e_{m,n}∈R¹ is the quantification error at pixel (m,n) and θ_{m,n}∈R¹ and \hat{θ}_{m,n}∈R¹ are the ground truth and estimated tissue properties at that pixel respectively. The relative error of an axial slice was calculated by averaging the relative errors at the pixels in the region of the brain. The mean and standard deviation of the relative errors of all testing slices were calculated for quantitative comparison between different methods.

One spatially-constrained tissue quantification method (SCQ) described herein was compared with the following existing methods for tissue quantification in MRF.

1) Baseline Method: The dictionary matching method proposed in the original MRF framework (DM) is selected as the baseline method for comparison.

### 2) State-of-the-Art Methods:

i) SDM: a variant of DM that uses SVD to compress the dictionary, which is reported to have better computation efficiency than DM,

ii) CSMR: a compressed-sensing-based method that uses multi-resolution reconstruction for MR images, which is reported to have good quantification accuracy for accelerated data with fewer time points,

iii) DL: a non-spatially-constrained deep-learning-based method, which is reported to have better computation efficiency than DM.

The SCQ method was compared with the baseline method (DM) for 3 acceleration rates:

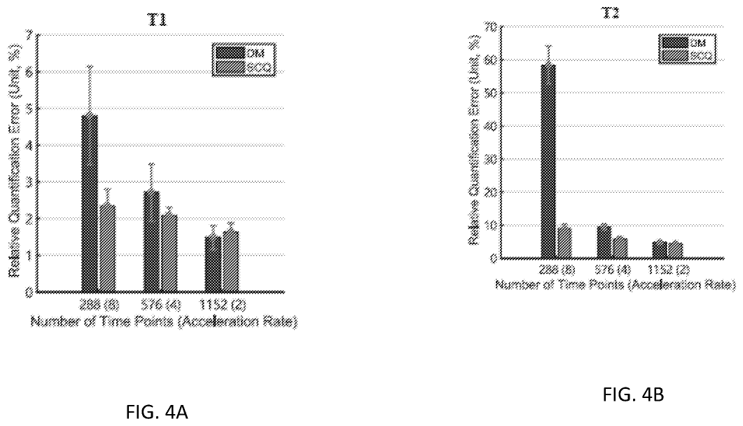

1) ar=2, T=1152; 2) ar=4, T=576; and 3) ar=8, T=288. The quantification results for a slice in test data yielded by the two methods confirm the SCQ method achieves more accurate quantification results than the baseline method in general. Notably, when ar=8, while DM completely fails to estimate the T2 map (error =60.9%), the SCQ method can still yield an accurate result for T2 (error=8.0%). The means and standard deviations of the relative errors of all slices in the test data are summarized in FIG. 4 As shown in FIG. 4, the SCQ method yields lower error than the baseline method for T2 quantification when ar=8, 4, and 2, and for T1 quantification when ar=8 and 4. Also, the advantage of the SCQ method is more significant when the acceleration rate is greater, i.e., when the acquisition time is shorter.

The SCQ method was also compared with the baseline and state-of-the-art methods in the terms of quantification accuracy and processing time. The experiments employed an ar=4. Subject-level leave-one-out cross validation was performed. Specifically, slices of 1 subject as the test data and slices of the remaining 5 subjects as the training data each time. Such process was repeated 6 times until all subjects were alternatively used as the test data. The quantification errors yielded by the competing methods for each test subject are summarized in Table I. As shown in Table I, the SCQ method consistently achieved the highest quantification accuracy in all the methods for quantification of T1 and T2.

TABLE 1
CROSS VALIDATION RESULTS

| | Subject | DM | SDM | CSMR | DL | SCQ (ours) |
|---|---|---|---|---|---|---|
| T1 | 1 | 2.39 ± 0.24 | 2.78 ± 0.29 | 2.61 ± 0.29 | 11.46 ± 2.14 | 1.87 ± 0.23 |
| | 2 | 2.40 ± 0.80 | 2.89 ± 0.93 | 2.65 ± 0.67 | 8.79 ± 2.56 | 1.83 ± 0.27 |
| | 3 | 2.75 ± 0.77 | 3.17 ± 0.86 | 2.91 ± 0.79 | 11.69 ± 2.39 | 2.07 ± 0.27 |
| | 4 | 2.90 ± 0.74 | 3.51 ± 0.97 | 4.38 ± 0.97 | 9.20 ± 1.74 | 1.85 ± 0.28 |
| | 5 | 2.71 ± 0.78 | 3.15 ± 0.96 | 3.34 ± 0.83 | 9.85 ± 2.17 | 2.08 ± 0.23 |
| | 6 | 2.13 ± 0.39 | 2.53 ± 0.58 | 2.29 ± 0.47 | 8.12 ± 1.53 | 1.64 ± 0.15 |
| | Overall | 2.55 ± 0.62 | 3.00 ± 0.76 | 3.03 ± 0.67 | 9.85 ± 2.09 | 1.89 ± 0.24 |
| T2 | 1 | 10.06 ± 0.75 | 13.99 ± 1.52 | 10.40 ± 0.93 | 12.35 ± 1.87 | 5.73 ± 0.71 |
| | 2 | 8.69 ± 0.68 | 11.87 ± 1.44 | 8.57 ± 0.59 | 13.25 ± 2.39 | 5.47 ± 0.60 |
| | 3 | 9.96 ± 1.52 | 13.96 ± 2.23 | 9.60 ± 1.43 | 12.77 ± 1.83 | 6.29 ± 0.89 |
| | 4 | 9.81 ± 0.58 | 14.16 ± 1.37 | 14.89 ± 4.91 | 12.91 ± 1.85 | 5.88 ± 0.56 |
| | 5 | 9.48 ± 0.84 | 13.46 ± 1.38 | 9.36 ± 0.65 | 11.73 ± 2.06 | 5.81 ± 0.66 |
| | 6 | 8.82 ± 0.87 | 13.34 ± 1.41 | 8.78 ± 1.57 | 11.39 ± 2.05 | 5.45 ± 0.70 |
| | Overall | 9.47 ± 0.87 | 13.46 ± 1.56 | 10.27 ± 1.68 | 12.40 ± 2.01 | 5.77 ± 0.69 |

The average processing times for an axial slice used by the competing methods are given in Table II. As shown in Table II, the SCQ method exhibited the shortest processing time among all methods, i.e. ˜1 second for quantification of T1 and T2 for an axial slice with 256×256 pixels.

TABLE II
PROCESSING TIME

| DM | SDM | CSMR | DL | SCQ (ours) |
|---|---|---|---|---|
| 9.18 s | 3.25 s | ~2 h | 4.93 s | 0.83 s |

### II. Methods of Generating Synthetic Magnetic Resonance Images

In another aspect, methods of generating synthetic magnetic resonance images employing quantitative magnetic resonance imaging data are described herein. In some embodiments, a method comprises identifying imaging parameters affecting tissue contrast for a type of magnetic resonance image and establishing Bloch equation simulations based on specific pulse sequence structure for the type of magnetic resonance image. Tissue intrinsic parameters are extracted from quantitative tissue maps acquired from a patient via a quantitative magnetic resonance imaging technique, and the synthetic magnetic resonance image is generated using the tissue intrinsic parameters in conjunction with the Bloch equation simulations. In some embodiments, the tissue intrinsic parameters and Bloch equation simulations are employed to simultaneously optimize all imaging parameters to achieve maximal contrast between the differing tissue types in the simulated imaging process employed to construct the synthetic magnetic resonance image. Tissue intrinsic parameters can include, but are not limited to, T1, T2 and spin density (M0). Moreover, the maximal contrast can be between healthy tissue and abnormal tissue.

Methods described in this Section II can be applied for MR imaging at all field strengths and scanners from different vendors. In the present example, MM measurements were performed on a Siemens 3T Prisma scanner using a 32-channel head coil. 3DMRF technique was used to measure T1, T2 and M0 maps, and the experiments were performed on both adult and pediatric subjects. While the MRF method was chosen due to its fast acquisition speed and high quantitative accuracy, other quantitative imaging methods can be used to acquire quantitative tissue maps for processing.

Based on the quantitative maps acquired using MRF, four different types of image contrasts including T1w, T2w, bSSFP, and DIR were synthesized. To demonstrate the key of optimized tissue contrast using brain images acquired from normal subjects, a workflow was developed with the purpose to optimize tissue contrasts between white matter and gray matter, as an example. First, all major imaging parameters that affect tissue contract were identified in each image type (T1w, T2w, bSSFP, or DIR), and the corresponding Bloch equation simulations were established based on its specific pulse sequence structure. Second T1, T2, and M0 values were extracted from white matter and gray matter for each scanned subject. Third, an optimization process was performed using the developed Bloch equation simulation and subject-specific tissue properties to optimize all imaging parameters simultaneously to achieve maximal tissue contrast.

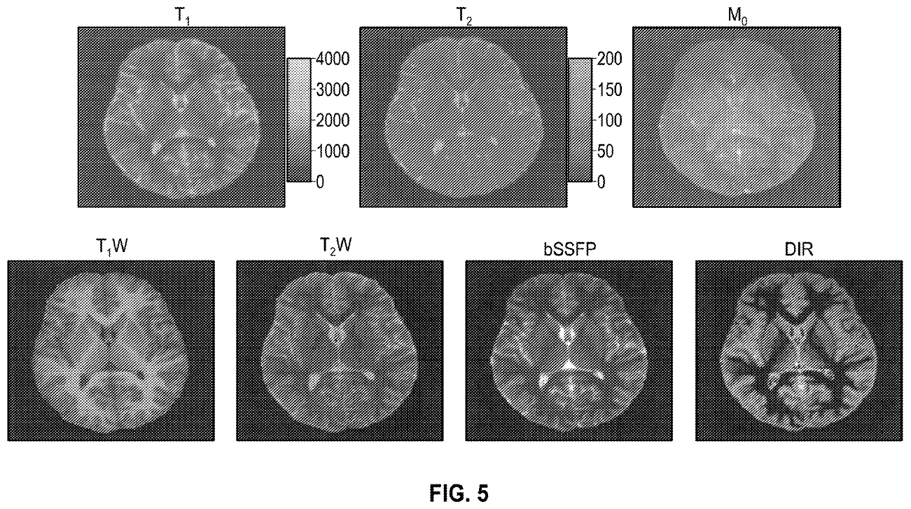

With the knowledge of quantitative tissue properties obtained using MRF from an adult subject, examples of synthetic images including T1w, T2w, bSSFP, and DIR images were generated, as illustrated in FIG. 5. All of the images were inherently co-registered, which facilitates direct comparison between contrasts.

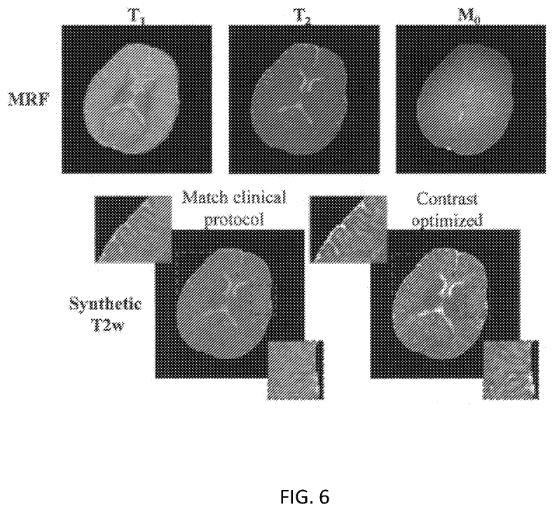

Based on the quantitative measures obtained from a 5-month-old pediatric subject, synthetic T2w images with optimized tissue contrasts between white matter and gray matter were generated. Both T1 and T2 values for white/gray matters were extracted from MRF measurement by a neuroradiologist and applied in Bloch equation simulations. Multiple imaging parameters in the T2w pulse sequence, such as echo time, echo train length, and 180° refocusing pulse design, were optimized to produce maximal contrasts between white and gray matters. The results demonstrate improved tissue contrasts as compared to the synthetic image generated bases on the standard imaging protocol, as illustrated in FIG. 6.

Compared to standard imaging methods, the development of optimized synthetic multiple contrast images according to systems and methods described herein is a technical advancement in at least the following aspect. (a) Maintaining tolerable acquisition time while obtaining multiple contrast synthetic images: With the intrinsic tissue parameters obtained from MRF, multi-contrast synthetic images can be generated (FIG. 1) without increasing data acquisition time. The available of multiple contract images can improve the ability to obtain detailed anatomical attributes for tissue characterization and lesion detection. (b) Inherently co-registered synthetic images: All synthetic images with different contrasts are inherently co-registered., which further facilitate multi-parametric analysis of abnormality in tissues. (c) Individually optimized image contrast: As disclosed above, optimization of imaging parameters tailored to a specific type of lesion or abnormality is practically impossible in clinical practice. However, with synthetic images, optimization can be done on the fly to generate the optimal contrast depending on the experimentally acquired tissue intrinsic parameters from each subject.

Although MRF was employed to obtain quantitative measures of tissue parameters in or to obtain synthetic images, systems and methods described in this Section II do not depend on MRF. Any approaches obtaining quantitative measures of tissue parameters can be used in such systems and methods.

### III. Systems and Methods of 3DMRF with Parallel Imaging

In a further aspect, methods of three-dimension magnetic resonance fingerprinting are described herein. Briefly, a method of three-dimensional magnetic resonance fingerprinting (MRF) comprises accelerating acquisition of a MRF dataset via application of parallel imaging along the partition-encoding direction, and integrating a convolutional neural network with MRF framework to extract an increased number of parameters from the MRF dataset yielding accelerated tissue mapping and one or more improvements to tissue characterization.

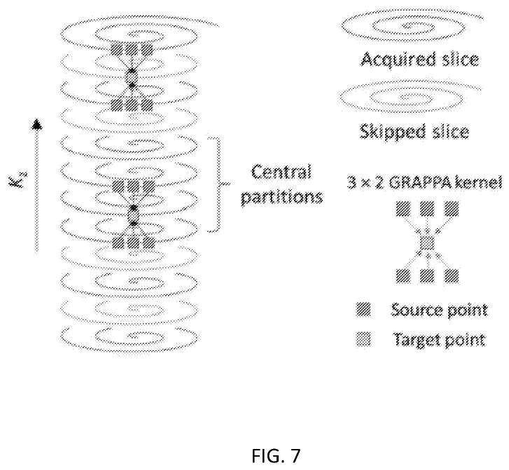

In this section, parallel imaging along the partition-encoding direction was applied to accelerate 3DMRF acquisition. An interleaved sampling pattern was used to undersample data in the partition direction. Parallel imaging reconstruction similar as the through-time spiral GRAPPA technique was applied to reconstruct the missing k-space points with a 3×2 GRAPPA kernel along the spiral readout×partition-encoding direction (FIG. 7). The calibration data for GRAPPA weight computation were obtained from the center k-space in partition direction and these calibration data were integrated in the final image reconstruction for preserved tissue contrast. One challenge for parallel imaging reconstruction with non-Cartesian trajectory, such as the spiral readout used in MRF acquisition, is to obtain sufficient repetition of the GRAPPA kernels for robust estimation of GRAPPA weights. Similar as the approach for the spiral GRAPPA technique, eight GRAPPA kernels with similar shape and orientation along the spiral readout direction were used in this study to increase the number of kernel repetitions. After GRAPPA reconstruction, each MRF time point/volume still has one spiral arm in-plane, but all the missing spiral arms along the partition direction are filled as illustrated in FIG. 7.

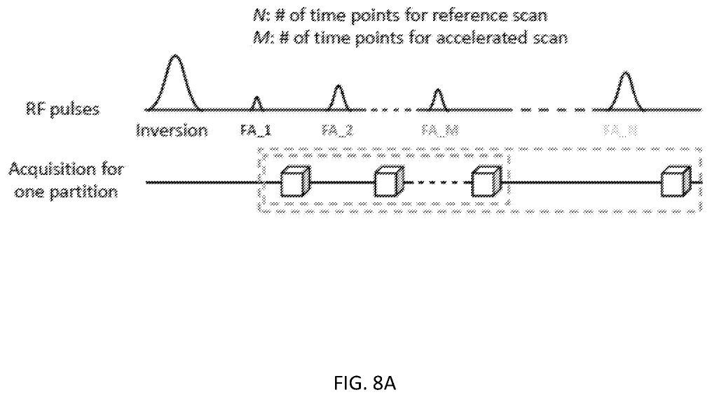

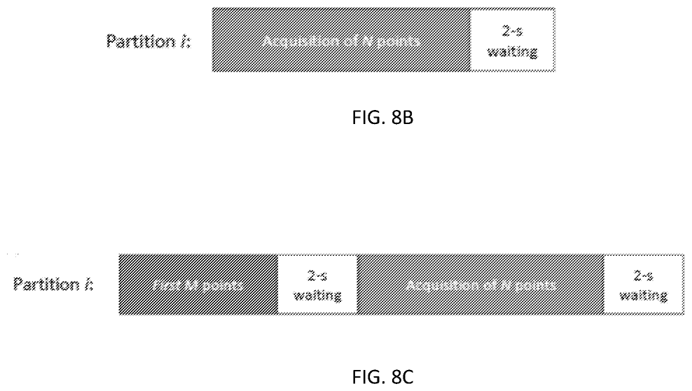

Besides acceleration with parallel imaging, the deep learning method was further leveraged to extract more features in the acquired MRF dataset to improve tissue characterization and reduce acquisition time. In order to describe the workflow for the application in 3DMRF, how deep learning is integrated into the 2DMRF framework is briefly reviewed. The ground truth tissue property maps (T₁ and T₂) are obtained using the template matching algorithm from MRF dataset consisting of N time frames (FIG. 8a). The purpose of accelerating MRF using deep learning is to achieve similar tissue map quality with only the first M time points (M<N). To train the CNN model for this purpose, the MRF signal evolution from M time points is used as the input of the CNN network and the output is the ground truth tissue maps obtained from all N points. To ensure data consistency between the network input and output and minimize potential motions in between, the input data of M points is generally obtained from retrospective undersampling of the reference data with all N points. For 2D measurements, it is reasonable to assume that each acquisition starts from a fully longitudinal recovered state (M_{z}=1). Therefore, the retrospectively undersampled MRF data and the data from prospectively accelerated case should have the same signal evolution for the same type of tissue. The CNN parameters determined in this manner can be directly applied to extract tissue properties from prospectively acquired dataset.

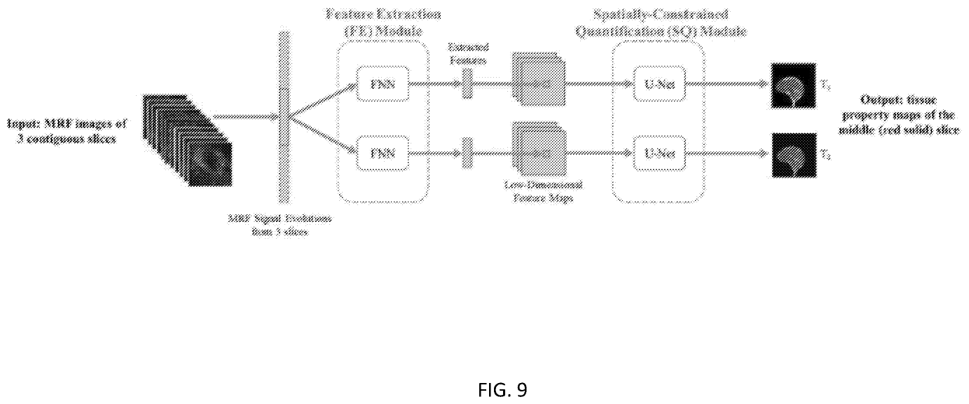

A CNN model with two major modules, a feature extraction module and a U-Net module, was used in the current study (FIG. 9). The feature extraction module consists of four fully-connected layers, which is designed to mimic singular value decomposition (SVD) to reduce the dimension of signal evolutions. While SVD functions as a single-layer linear mapping, the proposed feature extraction module provides a multilayer nonlinear mapping from the signal to the extracted features, which can be used to improve the robustness and accuracy of tissue quantification. The second U-Net module is used to capture spatial information of the feature map and finally generate the estimated tissue property. It is well-known that the performance of deep learning method is highly dependent on the number of convolutional layers and complexity of the network. U-Net has a contacting path and an expanding path, and it has a total of 23 convolutional layers with the standard structure. The network is designed to largely reduce the requirement of the size of training dataset.

While similar methods can be applied to reduce data sampling and accelerate 3DMRF, they face more challenges due to the additional partition encoding. For 3DMRF acquisition, a short waiting time (2 sec in this study) was applied between partitions (FIG. 8B), which is insufficient for most of brain tissues to achieve complete longitudinal recovery. As a result, the magnetization reached at the beginning of each partition acquisition is dependent on the settings acquiring the previous partition, including the number of MRF time frames. In this circumstance, the retrospectively shortened signal evolution with M time points (from a total of N time points) does not agree with the signal from prospectively accelerated scans and the CNN model trained in the aforementioned 2D approach is not applicable for the prospectively accelerated data. In order to train a CNN model for prospectively accelerated 3DMRF data, a new 3DMRF sequence was developed in this study and named 3DMRF for deep learning (3DMRF-DL).

The 3DMRF-DL sequence has a similar infrastructure as the standard 3DMRF method and acquires data sequentially along the partition direction. For data acquisition in each partition, an extra section was inserted to mimics the condition of the prospectively accelerated MRF acquisition (FIG. 8C). This additional section consists pulse sequence units for data sampling of the first M time points and 2-sec waiting time, which introduces the same magnetization history as the real accelerated case so that the data of the first M time points in the second section (containing all N time points) will match with the data in the actual accelerated scan. With this modification, data acquisition obtained in the second section of the 3DMRF-DL sequence can 1) provide reference T₁ and T₂ maps as the ground truth for CNN training and 2) generate retrospectively shortened MRF data (with M time points) as the input of training. Since the purpose of the additional section is to create magnetization history, no data acquisition is needed for this section.

Before the application of the developed 3DMRF-DL method for in vivo measurements, phantom experiments were performed using a phantom with MnCl₂ doped water to evaluate its quantitative accuracy. T₁ and T₂ values obtained using 3DMRF-DL were compared to those obtained with the reference methods using single-echo spin echo sequences and the standard 3DMRF method. Both the 3DMRF-DL and standard 3DMRF methods were conducted with 1-mm isotropic resolution and 48 partitions. The reference method was acquired from a single slice with FOV of 25 cm and matrix size of 128.

To use the 3DMRF-DL method to establish CNN for prospectively accelerated 3DMRF scans, the number of reduced time points M needs to be determined first. A testing dataset from five normal subjects (M:F, 2:3; mean age, 35±10 years) using the standard 3DMRF method was acquired for this purpose. The 3DMRF scan was performed with 1-mm resolution covering 96 partitions and 768 time points. Reference T₁ and T₂ maps were obtained using the template matching method and used as the ground truth for CNN network. To identify the optimum number of time frames, the CNN model was trained with various settings of input training data, with different M values (96, 144, 192 and 288) obtained with retrospective undersampling. Since the determination of optimum time frame is also coupled with the settings of parallel imaging, the extracted input data was also retrospectively undersampled along the partition direction with reduction factors of 2 or 3 and then reconstructed with parallel imaging. Dataset from four subjects were randomly selected for network training and the remaining dataset was used for validation. T₁ and T₂ maps obtained from various time points and reduction factors were compared to the ground truth maps and normalized root-mean-square-error (NRMSE) values were calculated to evaluate the performance and identify the optimum time point for the 3DMRF-DL method. One thing to note is that the CNN model training in this step cannot be applied to prospective accelerated data as introduced previously.

After the determination of the optimum time point for the accelerated scans, experiments were performed on seven normal volunteers (M:F, 4:3; mean age, 36±10 years) to establish the rapid 3DMRF method using parallel imaging and deep learning. For each subject, two separate scans were performed. The first scan was acquired using the 3DMRF-DL sequence with 144 slices. A total of 768 time points were acquired and no data undersampling was applied along the partition direction. For the second scan, standard 3DMRF sequence was used with prospective data undersampling, which includes sampling with reduced number of time points (M) and acceleration along the partition direction. Whole brain coverage (160˜176 saggital slices) was achieved for all the subjects. CNN model was then trained in the same approach as introduced above using the data acquired in the first scan. This trained model can be directly applied to extract T₁ and T₂ maps from the second prospectively accelerated scan. Cross-one validation was used to obtain T₁ and T₂ values from all seven subjects.

After tissue quantification using CNN, brain segmentation was further performed on both datasets to enable comparison of T₁ and T₂ values obtained from the two separate scans. To achieve this, T₁-weighted MPRAGE images were first synthesized based on the quantitative tissue properties maps. These MPRAGE images were used as the input and subsequent brain segmentation was performed using the Freesurfer software. Based on the segmentation results, mean T₁ and T₂ values from multiple brain regions, including white matter, cortical gray matter, subcortical gray matter and cerebrospinal fluid (CSF), were extracted for each subject and the results were compared between the two MRF scans.

A paired Student's t test was performed to compare the T₁ and T₂ values obtained using the 3DMRF-DL sequence and the prospectively accelerated 3DMRF sequence from different brain regions. A P value less than 0.05 was considered statistically significant in the comparisons.

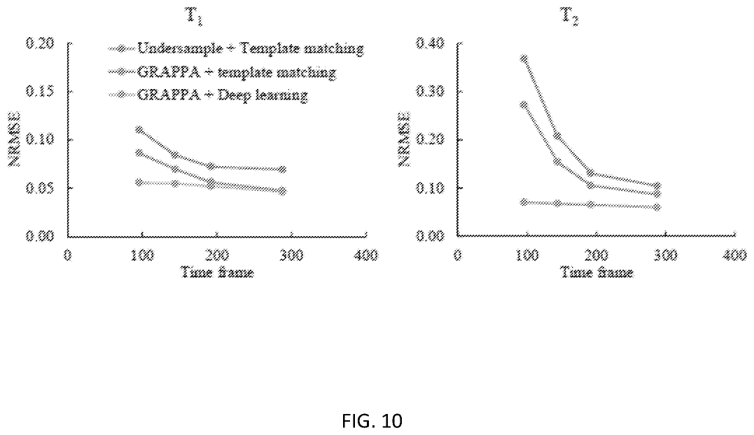

The standard 3DMRF sequence (FIG. 8B) was first applied to identify the optimum number of MRF time points and parallel imaging settings along the partition direction. MRF measurements from five subjects were retrospectively undersampled and reconstructed using parallel imaging and deep learning modeling. The results were further compared to those obtained using template matching alone or template matching after GRAPPA reconstruction. FIG. 4 shows representative results obtained from 192 time frames with a reduction factor of 2 in the partition-encoding direction. With 1-mm isotropic resolution, significant residual artifacts were noticed in both T₁ and T₂ maps processed with the template matching alone. With the GRAPPA reconstruction, most of artifacts in T₁ maps were eliminated, but some residual artifacts were still noticed in T₂ maps. Compared to these two approaches, the quantitative maps obtained with the proposed method with both GRAPPA reconstruction and deep learning modeling present similar quality as the reference maps. Lowest NRMSE values were obtained with the proposed method among all three methods. These findings are consistent for all other numbers of time points tested, ranging from 96 (12.5% of the total number of points acquired) to 288 (37.5%) as shown in FIG. 10.

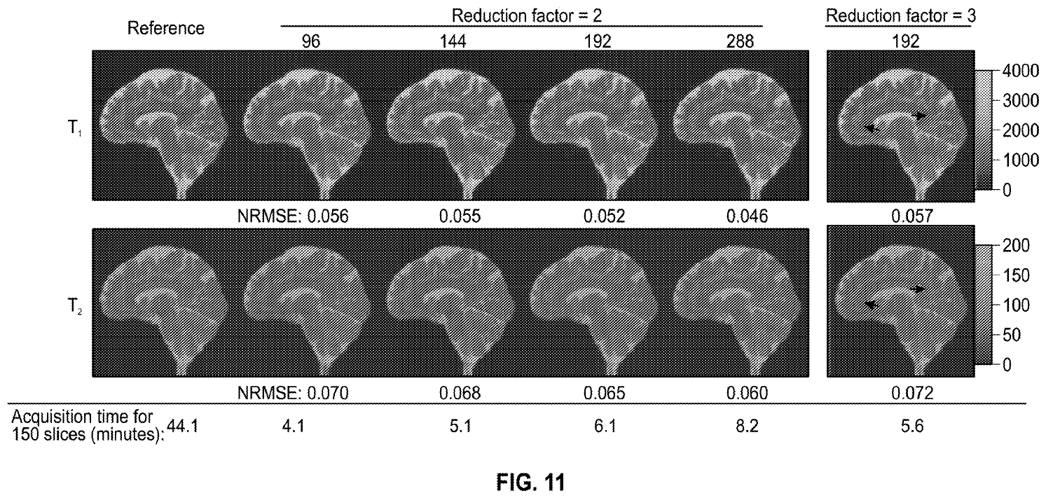

FIG. 11 shows representative T₁ and T₂ maps obtained using the proposed method with different numbers of time points. With the reduction factor of 2, high quality quantitative maps with 1-mm isotropic resolution were obtained for all the cases. When the number of time points increased, more information was utilized for tissue characterization and thus a decrease in NRMSE values was observed for both T₁ and T₂ maps. However, this improvement in tissue quantification was achieved at a cost of more sampling data and thus longer acquisition times. With the current design of 3DMRF sequence (R=2), the sampling time for 150 slices (15-cm coverage) was increased from 4.1 min to 8.2 min when the number of time frames increased from 96 to 288. Compared to the case with a reduction factor of 2, some residual aliasing artifacts were noticed in the quantitative maps obtained with the reduction factor of 3 (FIG. 11). In order to balance the image quality and scan time, a reduction factor of 2 with 192 time points was selected as the optimum setting for the following in vivo testing using the 3DMRF-DL approach.

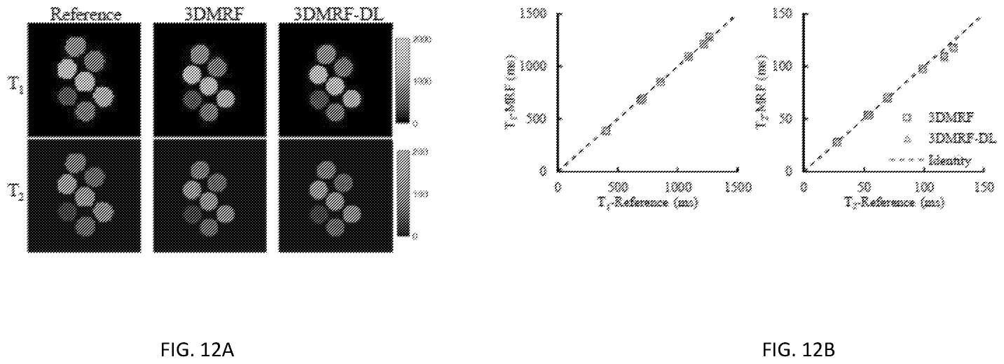

Before the application of the 3DMRF-DL sequence for in vivo measurements, its accuracy in T₁ and T₂ quantification was first validated using phantom experiments and the results are shown in FIG. 12. The T₁ and T₂ values obtained using the 3DMRF-DL method are consistent with the reference values for a wide range of T₁ from 400 to 1300 ms and T₂ from 30 and 130 ms. The percentage error averaged from all seven vials in the phantom was 1.7±2.2% and 1.3±2.9% for T₁ and T₂, respectively. The quality of the quantitative maps also matches well to the results acquired using the standard 3DMRF sequence. NRMSE value was 0.062 for T₁ and 0.046 for T2 between the results from two 3DMRF approaches.

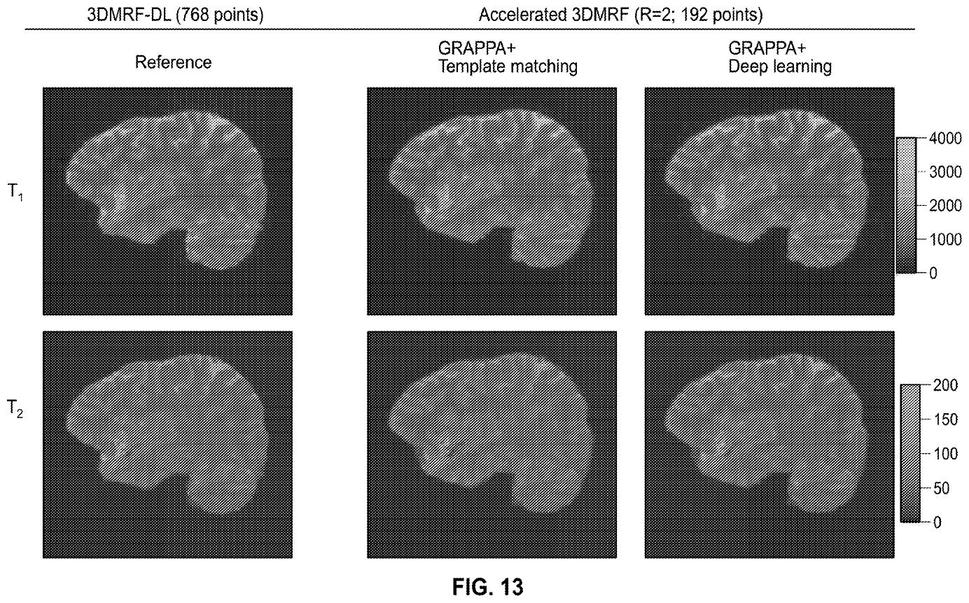

Based on the optimum time points (192 points) and undersampling patterns (R=2) determined in prior experiments, the 3DMRF-DL method was used to establish a CNN network for prospectively accelerated 3DMRF data. The experiments were performed on seven subjects and for each subject, two MRF scans, including one with all 768 time points using the 3DMRF-DL sequence and the other with only 192 points and prospectively accelerated 3DMRF sequence, were acquired. With the latter approach, about 160 to 176 slices were acquired for each subject to achieve whole-brain coverage and the acquisition time varies between 6.5 min and 7.1 min. Cross-one validation was performed to extract quantitative T₁ and T₂ values for all the subjects and the quantitative maps from both scans were calculated. Representative T₁ and T₂ maps obtained from the prospectively accelerated scan are presented in FIG. 13. Some residual artifacts are noticed in the images acquired with the GRAPPA+template matching approach, but removed with the proposed method combining GRAPPA with deep learning. The quantitative maps obtained from a similar slice location using the 3DMRF-DL method (served as the ground truth maps with all 768 time points) is also plotted for comparison (left column in FIG. 13). While relative head motions could exist between the two sets of results obtained from two separate scans, a good agreement in both brain anatomy and image quality were observed.

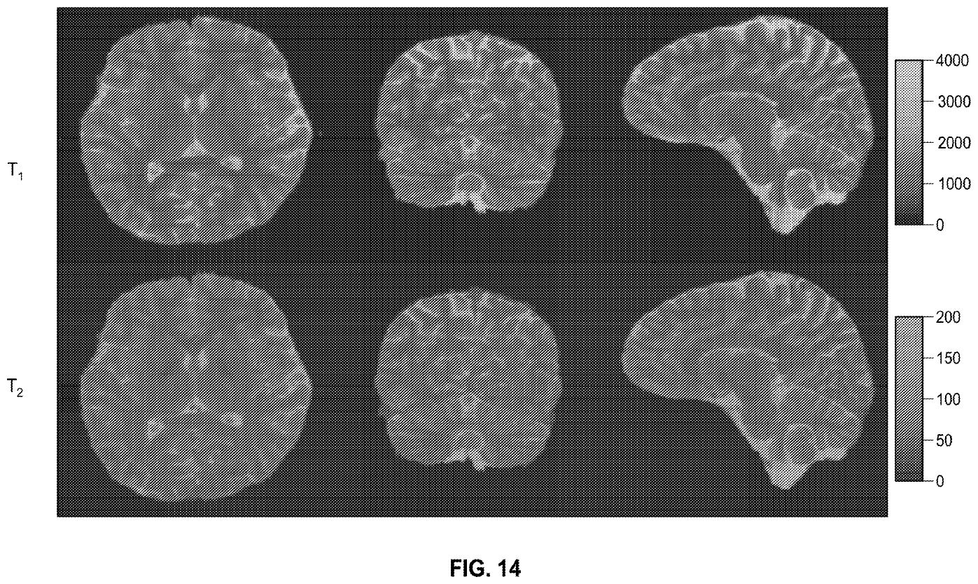

Representative T₁ and T₂ maps obtained using the accelerated scan from three different views are shown in FIG. 14. The results further demonstrate that high-quality 3DMRF with 1-mm resolution and whole-brain coverage can be achieved with the proposed approach in about 7 min. In addition, the time to extract tissue properties was also largely reduced to 2.5 sec/slice using the CNN method, which represents 7 fold of improvement as compared to the template matching method (˜18 sec/slice). While all these processing times were calculated based on computations performed on CPU, further acceleration in processing time can be achieved with direct implementation on a GPU card (0.02 sec/slice).

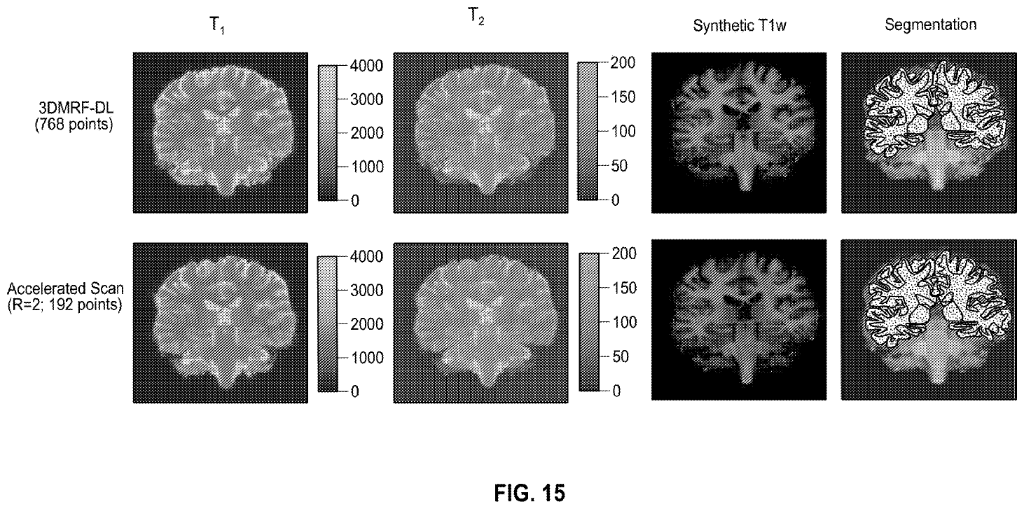

Representative segmentation results based on the MRF measurements are presented in FIG. 15. The quantitative T₁ and T₂ maps obtained using both the 3DMRF-DL sequence and the prospectively accelerated 3DMRF sequence are plotted, along with the synthetic T₁-weighted MPRAGE images and brain segmentation results. Different brain regions, such as white matter, gray matter, and thalamus, are illustrated with different colors in the maps and the segmentation results matched well between the two MRF scans.

In this application, a rapid 3DMRF method with a spatial resolution of 1 mm³ was developed, which can provide whole-brain (18-cm volume) quantitative T₁ and T₂ maps in ˜7 min. This is comparable to the acquisition time of conventional T₁-weighted and T₂-weighted images with a similar spatial resolution. By leveraging both parallel imaging and deep learning techniques, the proposed method demonstrates improved performance as compared to previously published methods. In addition, the processing time to extract T₁ and T₂ values was accelerated by more than 7 times with the deep learning approach as compared to the standard template matching method. Two advanced techniques, parallel imaging and deep learning, were combined to accelerate high-resolution 3DMRF acquisitions with whole brain coverage. The 3DMRF sequence employed in this study is already highly accelerated for in-plane encoding with only one spiral arm acquired (R=48). Therefore, more attention was paid to apply parallel imaging along the partition direction to further shorten the scan time. In addition, CNN has been shown to be capable of extracting more features from complex MRF signals in both spatial and temporal domains to improve tissue property mapping. This has been well demonstrated in previous 2D MRF studies. With 3D acquisitions, spatial constraints from all three dimensions were utilized for tissue characterization. The integration of advanced parallel imaging and convolutional neural networks provides complementary effects to 1) drastically reduce the amount of data needed for high-resolution MRF images and b) extract more advanced features, achieving improved tissue characterization and accelerated T₁ and T₂ mapping using MRF. Besides contributions to shorten MRF acquisitions in the temporal domain, the deep learning method also helps eliminate some residual artifacts in T₂ maps after the GRAPPA reconstruction. Recently, deep learning methods have been used for reconstruction of undersampled MR images and can achieve a higher acceleration factor as compared to conventional parallel imaging and compressed sensing techniques. However, the application of deep learning for non-Cartesian parallel imaging, such as spiral imaging, is limited and further developments in CNN methodologies will be conducted to address this problem in the future.

Parallel imaging along partition direction was applied to accelerate 3DMRF acquisition with 1-mm isotropic resolution. Results presented herein and others have shown that with such a high spatial resolution, the interleaved undersampling pattern with template matching does not resolve the aliasing artifacts in 3D imaging. By leveraging sliding window reconstruction, previous study has applied Cartesian GRAPPA to reconstruct 3DMRF dataset and a reduction factor of 3 was explored with the same spatial resolution. As described herein, advanced parallel imaging methods similar as spiral GRAPPA was used. To compute GRAPPA weights, the calibration data was acquired from the central partitions and integrated in the image reconstruction for preserved tissue contrast. This approach does not rely on the sliding window method, which could potentially reduce the MRF sensitivity along the temporal domain. With the proposed approach, high-quality quantitative T₁ and T₂ maps were obtained with a reduction factor of 2 and some artifacts were noticed with a higher reduction factor of 3. The difference at the higher reduction factor, as compared to findings in previous study, is likely due to different strategies to accelerate data acquisition. In this study, only 192 time points were acquired to form MRF signal evolution, while ˜420 points were used in the previous study. The more time points can be utilized to mitigate aliasing artifacts in the final quantitative maps, but at a cost of longer sampling time for each partition.

A modified 3DMRF-DL sequence was developed to acquire the necessary dataset to train the CNN model that can be applied to prospectively accelerated 3DMRF data. With the standard 3DMRF sequence, a short waiting time (typically 2˜3 sec) was applied between the acquisitions of different partitions for longitudinal relaxation. Due to the incomplete T₁ relaxation with this short waiting time, the retrospectively shortened dataset acquired with this sequence does not match the prospectively acquired accelerated data even with the same number of time points. One potential method to mitigate this problem is to acquire two separate scans, one accelerated scan with reduce time points and the other with all N points to extract ground truth maps. However, considering the long scan time to obtain the ground truth maps, this method is sensitive to subject motions between scans and even a small motion between the MRF images and the corresponding tissue property maps could potentially lead to incorrect estimation of parameters in the CNN model. Image registration can be applied to correct relative motions between scans, but variations could be introduced during the registration process. The proposed 3DMRF-DL method provides an alternative solution for this issue and generates necessary data without the concern of relative motion in the CNN training dataset. While extra scan time is needed with the additional pulse sequence section, the total acquisition is the same as the means to acquire two separate scans to solve the issue.

In the proposed 3DMRF-DL sequence, a preparation module containing the pulse sequence section for the first M time points was added before the actual data acquisition section. One potential concern is whether one preparation module will be sufficient to generate the same spin history as the prospectively accelerated scans. Previous studies have shown that when computing the dictionary for 3DMRF, simulation with one such preparation module is sufficient to reach the magnetization state for calculation of the MRF signal evolution in the actual acquisitions. Simulation results have also shown that the signal evolution obtained from the proposed 3DMRF-DL method matched well with the prospectively accelerated 3DMRF method. All these findings suggest that the one preparation module added in the 3DMRF-DL sequence is sufficient to generate the magnetization state as needed.

Subject motion in clinical imaging presents one of the major challenges for high-resolution MR imaging. Compared to the standard MR imaging with Cartesian sampling, MRF utilizes a non-Cartesian spiral trajectory for in-plane encoding, which is known to yield better performance in the presence of motion. The template matching algorithm used to extract quantitative tissue properties also provides a unique opportunity to reduce motion artifacts. As demonstrated in the original 2DMRF paper, the motion-corrupted time frames behave like noise during the template matching process and accurate quantification was obtained in spite of subject motion. However, the performance of 3DMRF in the presence of motion has not been fully explored. A recent study has shown that 3DMRF with linear encoding along partition-encoding direction is also sensitive to motion artifacts, and the degradation in T₁ and T₂ maps is likely dependent on the magnitude and timing of the motion during the 3D scans. The 3DMRF approach described herein will help reduce motion artifacts with the accelerated scans. The lengthy acquisition of training dataset acquired in this study is more sensitive to subject motion. While no evident artifacts were noticed with all subjects scanned in this study, further improvement in motion robustness is needed for 3DMRF acquisitions.

As described herein a high-resolution 3D MR Fingerprinting technique, combining parallel imaging and deep learning, was developed for rapid and simultaneous quantification of T₁ and T₂ relaxation times. Our results show that with the integration of parallel imaging and deep learning techniques, whole-brain quantitative T₁ and T₂ mapping with 1-mm isotropic resolution can be achieved in ˜7 min, which is feasible for routine clinical practice.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A system for tissue quantification in magnetic resonance fingerprinting (MRF) comprising:
a feature extraction module operable to convert pixel input high-dimensional signal evolution in an axial slice to a low-dimensional feature map; and
a spatially constrained quantification module operable to capture spatial information from the low-dimensional feature map and generate an estimated tissue property map.

2. The system of claim 1, wherein the feature extraction module is applied to all pixels in an axial slice to generate the low-dimensional feature map.

3. The system of claim 1, wherein the feature extraction module employs a fully-connected neural network to convert the high-dimensional signal evolution into the low-dimensional feature vector.

4. The system of claim 3, wherein the fully-connected neural network comprises one or more fully connected layers, each fully connected layer having a linear projection followed by batch normalization and ReLU normalization.

5. The system of claim 1, wherein the spatially constrained quantification module employs a convolutional neural network to capture the spatial information.

6. The system of claim 1, wherein time points for acquisition of the input high-dimensional signal evolution is reduced by at least 50 percent.

7. The system of claim 1, wherein time points for acquisition of the input high-dimensional signal evolution is reduced by at least 75 percent.

8. The system of claim 1, wherein the estimated tissue property map is a T1 map.

9. The system of claim 1, wherein the estimated tissue property map is a T2 map.

10. A method for tissue quantification in magnetic resonance fingerprinting (MRF) comprising:
providing pixel input high-dimensional signal evolution to a feature extraction module to generate a low-dimensional feature map; and
transferring the low dimension feature map to a spatially constrained quantification module for capturing spatial information from the low-dimensional feature map and generating an estimated tissue property map.

11. A method of generating a synthetic magnetic resonance image of tissue comprising:
identifying imaging parameters affecting tissue contrast for a type of magnetic resonance image;
establishing Bloch equation simulations based on specific pulse sequence structure for the type of magnetic resonance image;
extracting tissue intrinsic parameters of differing tissue types from quantitative tissue maps acquired from a patient via a quantitative magnetic resonance imaging technique; and
generating the synthetic magnetic resonance image using the tissue intrinsic parameters in conjunction with the Bloch equation simulations.

12. The method of claim 11, wherein the tissue intrinsic parameters and Bloch equation simulations are employed to simultaneously optimize all imaging parameters to achieve maximal contrast between the differing tissue types in the synthetic magnetic resonance image.

13. The method of claim 11, wherein the type of magnetic resonance image is selected from the group consisting of T1-weighted (T₁W), T2-weighted (T₂W), fluid-attenuated inversion recovery (FLAIR), steady-state free precession (SSFP) and double inversion recovery (DIR).

14. The method of claim 11, wherein the tissue intrinsic parameters are T1, T2 and spin density (M0).

15. The method of claim 12, wherein the maximal contrast is between healthy tissue and abnormal tissue.

16. A method of three-dimensional magnetic resonance fingerprinting (MRF) comprising:
accelerating acquisition of a MRF dataset via application of parallel imaging along the partition-encoding direction; and
integrating a convolutional neural network with MRF framework to extract an increased number of parameters from the MRF dataset yielding accelerated tissue mapping and one or more improvements to tissue characterization.

17. The method of claim 16 having a spatial resolution of 1 mm³.
